(19) United States
(12) Patent Application Publication
(10) Pub. No.: US 2025/0312249 A1
(43) Pub. Date: Oct. 9, 2025

(54) COMPOSITIONS AND METHODS FOR ALTERING THE COLOR OF HAIR

(71) Applicant: L'Oreal, Paris (FR)

(72) Inventors: Sarah Mary SPEER, Linden, NJ (US); Tathagata MUKHERJEE, Union, NJ (US); Shahid Mohammad NASEER, Hasbrouck Heights, NJ (US)

(21) Appl. No.: 18/626,294

(22) Filed: Apr. 3, 2024

(51) Int. Cl. A61K 8/26; A61Q 5/08

(52) U.S. Cl. CPC A61K 8/26; A61Q 5/08; A61K 2800/30; A61K 2800/805

(57) ABSTRACT

The disclosure relates to compositions and methods for altering the color of hair, the compositions comprising at least one color-removing agent chosen from clay compounds and at least one solvent.

### TECHNICAL FIELD

The disclosure relates to compositions and methods for altering the color, tone, and/or shade of hair. The compositions and methods can be used to alter the color of hair by removing various color, tones, and/or shades from the hair.

### BACKGROUND

Consumers often desire to change the color of their hair. For example, as a person ages, the hair follicle loses its natural pigment, resulting in grey, silver, or white hair. It is common for consumers to color their grey hairs, for example to match their natural hair color. Similarly, consumers may wish to change the color, tone, and/or shade of their hair from that of their natural color or a previous hair dye, e.g. may wish change to a different color entirely, may wish to lighten or darken their hair color, may wish to add warmth or coolness to the color of their hair, etc. The most common hair dyeing processes are permanent, semi-permanent, and temporary hair dyeing.

Permanent hair dyeing compositions uses oxidation dye precursors, which are also known as primary intermediates or couplers. In order to provide various colors, tones, and shades of permanent hair color, dye compositions contain various combinations of different types of oxidation dye compounds. These oxidation dye compounds are small, colorless or weakly colored compounds which, when combined with oxidizing agents, give rise to colored complexes by a process of oxidative condensation. The permanent hair dye compositions also contain ammonia or other alkalizing agents which causes the hair shaft to swell, thus allowing the oxidative dye molecules to penetrate the cuticle and cortex before the oxidation condensation process is completed. The resulting larger-sized colored complexes from the oxidative reaction are then trapped inside the hair fiber and cannot be washed out, thereby permanently altering the color of the hair.

On the other hand, temporary hair dye compositions typically use pigments, liposoluble dyes, or direct dyes which are deposited onto the hair fiber to impart color to the hair. Temporary hair dyes are washed out of the hair after one or two shampoo cycles.

In between permanent and temporary dyes are semi-permanent hair dyes. Semi-permanent dyes can remain longer than temporary dyes, but not as long as permanent dyes, typically lasting from four to twelve shampoo cycles. While semi-permanent dyes are ideal for altering the color of the hair on a lasting but non-permanent basis, these products present problems in the case where an individual wishes to change the hair color before it fades over time. For example, if an individual who has previously changed the color or tone of their hair with a first semi-permanent dye composition wishes to alter the color or tone of their previously-colored hair, the first dye must be either removed or neutralized so that it no longer imparts the first color to the hair fiber.

If an individual who has previously changed the color or tone of their hair with a semi-permanent dye composition wishes to alter the color or tone of their previously-colored hair, some or all of the color imparted by the first semi-permanent dye composition must be either removed or neutralized so that it no longer imparts the first color to the hair fiber. For example, a consumer may wish to remove the first color or tone in order to go back to their natural hair color, or to achieve a second color different from the first color by using a second dye composition. In such cases it is necessary to remove all of the first color from the hair, either to allow the natural hair color to be visible or to prevent the first color from interfering with the appearance of the second color. As another example, a consumer may wish to achieve a second tone or shade in the same color family as the first color, such as changing hair color from dark brown to light brown, and therefore may desire to remove some, but not necessarily all, of the first color. Alternatively, the change desired may be more subtle, for example by increasing the warmth or brightness of the first color. Thus, depending on the particular combination of dye compounds present in the first dye composition, and the desired second color or tone to be achieved, different levels and types of color removal are required.

Although there are known methods that are effective for removing the first color from the hair, these methods have drawbacks. For example, one common color-removal process uses oxidizing agents, but these compositions are harsh and lead to damage to the hair, causing the hair to be weak, brittle, and have an unhealthy appearance. In view of the damaged caused by these compositions, they are not typically used for removing semi-permanent hair dyes. Moreover, these compositions can remove essentially all color from the hair, and therefore subsequent application of a second dye composition is needed to achieve the desired second color or tone. This step, known as “backfilling” the color, can further expose the hair to harsh chemicals and lead to additional damage.

A second option involves the use of compositions with reducing agents such as thiol-based compounds. However, these compositions are not very effective at removing semi-permanent hair dyes; as such, the color result achieved may not be satisfactory when used on hair that has been dyed with such compounds, and the remaining color will interfere with the colorist's attempt to achieve the desired color, tone, or shade.

Further, if an individual wishes to remove only semi-permanent dyes without also removing permanent (oxidative) dyes, neither oxidizing agents nor thiol-based color removing agents can achieve the desired result since both are known to remove oxidative dye from hair.

It has now been surprisingly discovered that compositions comprising clay compounds permit controlled removal of semi-permanent dyes from previously-dyed hair, providing desired alterations in hair color, tone, and/or shade. The compositions are unique in that they can thus be used in color-removal processes to achieve a desired color, tone, and/or shade, much like consumers experience when undergoing a hair dye process, even in the absence of a backfilling step.

### SUMMARY

The disclosure relates to compositions and methods that can be used to remove artificial color from the hair, in particular hair that has previously been dyed with a semi-permanent dye composition. In various embodiments, the compositions and methods can remove some or all of certain dyes, while having minimal or no impact on other dyes. As such, the color left on the hair can be “tuned” to achieve the consumer's desired result. The compositions and methods are able to achieve a desired hair color, tone, and/or shade even without, or with minimal, application of, subsequent hair coloring compositions.

In various embodiments, the disclosure relates to compositions for treating keratin fibers such as hair, for example for removing color from the hair, the compositions comprising (a) at least one color-removing agent chosen from clay compounds, and (b) at least one solvent. The compositions optionally comprise at least one additional component chosen from surfactants, fatty compounds, thickeners, amino acids and/or salts thereof, amino-sulfonic acids and/or salts thereof, or combinations of two or more thereof. The solvent may be chosen from water and/or non-aqueous solvents. The pH of the compositions may range from about 5 to about 11, such as from about 6 to about 10, from about 6.5 to about 9.5, from about 7 to about 9, from about 7.5 to about 8.5, or may be about 8.

By way of non-limiting example, the clay compounds may be chosen from kaolin, hectorite, magnesium aluminum silicate, bentonite, or combinations of two or more thereof. In various embodiments, the total amount of clay compounds in the composition can range from about 0.1% to about 30%, such as from about 0.5% to about 25%, from 1% to about 20%, or from about 5% to about 15% by weight, relative to the total weight of the composition. In some embodiments, the composition comprises no more than 0.5% of color-removing agents other than clay compounds. In other embodiments, the compositions are free or essentially free of other color removing components such as oxidizing agents and/or reducing agents, and free or essentially free of alkalizing agents.

In further embodiments, the disclosure relates to methods of treating hair using the compositions according to the disclosure. For example, the methods may be methods of removing semi-permanent dyes from the hair. The methods may also be methods of modifying the color, tone, and/or shade of the hair. In some embodiments, a composition according to the disclosure may be applied to the hair and left on the hair for a period of time, for example ranging from about 10 minutes to about 6 hours, from about 30 minutes to about 3 hours, from about 45 minutes to about 150 minutes, from about 60 minutes to about 2 hours, etc. The hair may optionally be heated during the leave-in period. The methods may further comprise removing the compositions from the hair, e.g. by rinsing and/or shampooing the hair.

### DETAILED DESCRIPTION

The disclosure relates to compositions and methods for removing semi-permanent dyes from hair. The methods comprise removing some or all of various dye compounds in order to provide a desired color, tone, and/or shade of the treated hair.

### I. Compositions

Compositions according to the disclosure comprise (a) at least one color-removing agent chosen from clay compounds, and (b) at least one solvent, and may optionally include at least one additional compound chosen from surfactants, fatty compounds, thickeners, amino acids, or combinations of two or more thereof.

### Clay Compounds

The compositions comprise at least one clay compound. It has surprisingly been discovered that clay compounds are effective at removing semi-permanent hair dyes, even in the absence of traditional hair color removing agents such as oxidizing agents and reducing agents.

By way of non-limiting example, the clay compound may be chosen from kaolinite (also referred to interchangeably as kaolin), bentonite, magnesium aluminum silicate, hectorite, smectite, vermiculite, illite, chorite, halloysite, palygorskite, sepiolite, sesquioxide, imogolite, allophane, or combinations of two or more thereof. For example, in one embodiment, kaolin is chosen. In another embodiment, bentonite is chosen. In various embodiments, the clay compound comprises, consists essentially of, or consists of smectite, hectorite, kaolin, bentonite, magnesium aluminum silicate, or a combination of two or more thereof.

If present, the total amount of clay can range from about 0.1% to about 30%, such as from about 0.5% to about 25%, from about 1% to about 20%, or from about 2% to about 18% by weight, relative to the total weight of the composition. For example, the total amount of clay may range from about 1% to about 20%, from about 1% to about 19%, from about 1% to about 18%, from about 1% to about 17%, from about 1% to about 16%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 20%, from about 2% to about 19%, from about 2% to about 18%, from about 2% to about 17%, from about 2% to about 16%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 20%, from about 3% to about 19%, from about 3% to about 18%, from about 3% to about 17%, from about 3% to about 16%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 3% to about 4%, from about 4% to about 20%, from about 4% to about 19%, from about 4% to about 18%, from about 4% to about 17%, from about 4% to about 16%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 20%, from about 5% to about 19%, from about 5% to about 18%, from about 5% to about 17%, from about 5% to about 16%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6% by weight, relative to the total weight of the composition. For example, the total amount of clays may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the composition, including any range using any of the foregoing as upper and lower limits.

As a non-limiting example, the clay compound may comprise, consist essentially of, or consist of magnesium aluminum silicate, bentonite, kaolin, smectite, hectorite, or a combination of two or more thereof, and the total amount of clays may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the composition, or may be present in a range using any of the foregoing as upper or lower limits.

### Solvents

Compositions according to the disclosure comprise at least one solvent. The solvent may be chosen from water, non-aqueous solvents, or combinations thereof.

In some embodiments, the solvent comprises, consists essentially of, or consists of water. The total amount of water may vary depending on the desired properties of composition, for example consistency, viscosity, etc.

In some embodiments, non-aqueous solvents may be used, for example, glycerin, C₁₋₄ alcohols, fatty alcohols, fatty ethers, fatty esters, polyols, glycols, vegetable oils, mineral oils, liposomes, laminar lipid materials, or combinations thereof. Non-limiting examples of non-aqueous solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

The total amount of solvents in the composition typically ranges from about 50% to about 98%, such as from about 60% to about 98%, from about 70% to about 98%, from about 75% to about 98%, from about 75% to about 95%, or from about 80% to about 90% by weight, relative to the total weight of the composition. In some embodiments, the solvent comprises, consists essentially of, or consists of water. For example, in some embodiments the solvent is at least about 75%, such as at least about 80%, at least about 85%, at least about 90%, at least about 95%, at least about 96%, at least about 97%, at least about 98%, or at least about 99% water by weight, relative to the total solvent.

### Surfactants

Compositions according to the disclosure optionally comprise at least one surfactant, which may in various embodiments be chosen from anionic, non-ionic, amphoteric, and/or cationic surfactants. For example, the compositions may comprise one or more anionic surfactants, one or more non-ionic surfactants, or one or more amphoteric surfactants, one or more cationic surfactants, or the compositions may comprise mixtures of surfactants having the same or different ionicities. Salts of surfactants are included whether or not expressly stated, unless specified otherwise.

### Anionic Surfactants

In at least some embodiments, the compositions comprise at least at least one anionic surfactant. The term “anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from, for example, —CO₂H, —CO₂⁻, —SO₃H, —SO₃⁻, —OSO₃H, —OSO₃⁻, —H₂PO₃, —HPO₃⁻, —PO₃²⁻, —H₂PO₂, ═HPO₂, —HPO₂⁻, ═PO₂⁻, ═POH, and ═PO⁻ groups.

The anionic surfactants may be sulfate, sulfonate, and/or carboxylic (or carboxylate) surfactants, or mixtures thereof.

Sulfate anionic surfactants comprise at least one sulfate function (—OSO₃H or —OSO₃) but generally do not comprise any carboxylate or sulfonate functions. In some embodiments the sulfate anionic surfactants that are used are free of carboxylate or sulfonate functions.

Non-limiting examples of sulfate anionic surfactants include alkyl sulfates, alkyl ether sulfates, alkylamido ether sulfates, alkylaryl polyether sulfates, monoglyceride sulfates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, for example polyoxyethylenated, and optionally comprising from 1 to 50 ethylene oxide units such as from 2 to 10 ethylene oxide units.

Sulfonate anionic surfactants comprise at least one sulfonate function (—SO₃H or —SO₃⁻) and may optionally also comprise one or more sulfate functions, but preferably do not comprise any carboxylate functions.

Non-limiting examples of sulfonate anionic surfactants include alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds; the alkyl groups of these compounds comprising from 6 to 30 carbon atoms, for example from 12 to 28, such as from 14 to 24 or even from 16 to 22 carbon atoms; the aryl group preferably denoting a phenyl or benzyl group; these compounds possibly being polyoxyalkylenated, preferably polyoxyethylenated, and optionally comprising from 1 to 50 ethylene oxide units and better still from 2 to 10 ethylene oxide units.

Carboxylate anionic surfactants comprise at least one carboxylic or carboxylate function (—COOH or —COO⁻) and may optionally also comprise one or more sulfate and/or sulfonate functions.

Non-limiting examples of carboxylate anionic surfactants include acylglycinates, acyllactylates, acylsarcosinates, acylglutamates, alkyl-D-galactosideuronic acids, alkyl ether carboxylic acids, alkyl(C6-30 aryl) ether carboxylic acids, alkylamido ether carboxylic acids; and also the salts of these compounds; the alkyl and/or acyl groups of these compounds comprising from 6 to 30 carbon atoms, for example from 12 to 28, such as from 14 to 24 or even from 16 to 22 carbon atoms; the aryl group preferably denoting a phenyl or benzyl group; these compounds possibly being polyoxyalkylenated, preferably polyoxyethylenated, and optionally comprising from 1 to 50 ethylene oxide units and better still from 2 to 10 ethylene oxide units.

When the anionic surfactant is in salt form, the salt may be chosen from alkali metal salts, such as the sodium or potassium salt, ammonium salts, amine salts and in particular amino alcohol salts, and alkaline-earth metal salts, such as the magnesium salt.

Examples of amino alcohol salts may include but are not limited to monoethanolamine, diethanolamine, and triethanolamine salts, monoisopropanolamine, diisopropanolamine, and triisopropanolamine salts, 2-amino-2-methyl-1-propanol salts, 2-amino-2-methyl-1,3-propanediol salts, and tris(hydroxymethyl)aminomethane salts.

In some embodiments, alkali metal or alkaline-earth metal salts and in particular the sodium or magnesium salts may be chosen, for example in the form of alkali metal, ammonium, amino alcohol and alkaline-earth metal salts, or a mixture of these compounds, may be chosen.

In exemplary and non-limiting embodiments, the anionic surfactant may be chosen from sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C₁₄₋₁₆ Olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, sodium cocoyl glutamate, disodium cocoyl glutamate, potassium myristoyl glutamate, TEA-cocoyl glutamate, sodium cocoyl glycinate, potassium cocoyl glycinate, sodium cocoyl alaninate, TEA-cocoyl alaninate, or a combination of two or more thereof. For example, the compositions may comprise at least one anionic surfactant chosen from sodium laureth sulfate, sodium lauryl sulfate, sodium lauroyl sulfate, sodium lauroyl methyl isethionate, or a combination of two or more thereof.

If present, the total amount of anionic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of anionic surfactants may range from about 0.01% to about 12%, from about 0.1% to about 10%, from about 0.5% to about 8%, or from about 1% to about 6% by weight, relative to the total weight of the composition. In at least some embodiments, the compositions comprise at least one anionic surfactant, and have a total amount of anionic surfactants ranging from about 0.5% to about 10%, from about 0.5% to about 8%, from about 0.5% to about 6%, from about 0.5% to about 5%, from about 0.5% to about 4%, from about 0.5% to about 3%, from about 0.75% to about 10%, from about 0.75% to about 8%, from about 0.75% to about 6%, from about 0.75% to about 5%, from about 0.75% to about 4%, from about 0.75% to about 3%, from about 1% to about 10%, from about 1% to about 8%, from about 1% to about 6%, from about 1% to about 5%, from about 1% to about 4%, from about 1% to about 3%, from about 1.25% to about 10%, from about 1.25% to about 8%, from about 1.25% to about 6%, from about 1.25% to about 5%, from about 1.25% to about 4%, from about 1.25% to about 3%, from about 1.5% to about 10%, from about 1.5% to about 8%, from about 1.5% to about 6%, from about 1.5% to about 5%, from about 1.5% to about 4%, or from about 1.5% to about 3%, by weight relative to the total weight of the composition. In various embodiments, the total amount of anionic surfactant may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, or may be present in any range using any of the foregoing as upper and lower limits.

### Amphoteric Surfactants

In at least some embodiments, the compositions comprise at least one amphoteric (also referred to as zwitterionic surfactants), although in at least some embodiments the compositions are free or essentially free of amphoteric surfactants. Non-limiting examples of useful amphoteric surfactants include derivatives of aliphatic secondary and tertiary amines where the aliphatic radical can be straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphohydroxypropylsulfonate, triethanolamine cornamphopropionate, cocoamphoacetate, triethanonlamine triethanonlamine cocoamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine, as well as combinations of two or more thereof.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, oleyl betaine, cocamidopropyl betaine, or combinations of two or more thereof, may be chosen.

If present, the total amount of amphoteric surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of amphoteric surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one amphoteric surfactant, and have a total amount of amphoteric or zwitterionic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In at least some other embodiments, the compositions are free or substantially free of amphoteric surfactants.

### Nonionic Surfactants

In at least some embodiments, the compositions comprise at least one nonionic surfactant, although in at least some embodiments the compositions are free or essentially free of nonionic surfactants. The nonionic surfactants may be chosen from alcohols, α-diols and (C₁-C₂₀) alkylphenols, these compounds being polyethoxylated, polypropoxylated and/or polyglycerolated, the number of ethylene oxide and/or propylene oxide groups possibly ranging from 1 to 100, and the number of glycerol groups possibly ranging from 2 to 30, or alternatively these compounds comprising at least one fatty chain comprising from 8 to 30 carbon atoms and especially from 16 to 30 carbon atoms. For example, nonionic surfactants may be chosen from monooxyalkylenated or polyoxyalkylenated (C₈-C₂₄) alkylphenols, saturated or unsaturated, linear or branched, monooxyalkylenated or polyoxyalkylenated C₈-C₃₀ alcohols, saturated or unsaturated, linear or branched, monooxyalkylenated or polyoxyalkylenated C₈-C₃₀ amides, esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol, saturated or unsaturated, monooxyalkylenated or polyoxyalkylenated plant oils, condensates of ethylene oxide and/or of propylene oxide, or combinations thereof.

By way of example only, the adducts of ethylene oxide with lauryl alcohol, for example those containing from 9 to 50 oxyethylene units or from 10 to 12 oxyethylene units (Laureth-10 to Laureth-12); the adducts of ethylene oxide with behenyl alcohol, for example those containing from 9 to 50 oxyethylene units (Beheneth-9 to Beheneth-50); the adducts of ethylene oxide with cetearyl alcohol (mixture of cetyl alcohol and stearyl alcohol), for example those containing from 10 to 30 oxyethylene units (Ceteareth-10 to Ceteareth-30); the adducts of ethylene oxide with cetyl alcohol, for example those containing from 10 to 30 oxyethylene units (Ceteth-10 to Ceteth-30); the adducts of ethylene oxide with stearyl alcohol, for example those containing from 10 to 30 oxyethylene units (Steareth-10 to Steareth-30); the adducts of ethylene oxide with isostearyl alcohol, for example those containing from 10 to 50 oxyethylene units (Isosteareth-10 to Isosteareth-50); monoglycerolated or polyglycerolated C₈-C₄₀, e.g. C₈-C₃₀, alcohols, such as lauryl alcohol containing 4 mol of glycerol (Polyglyceryl-4 Lauryl Ether), lauryl alcohol containing 1.5 mol of glycerol, oleyl alcohol containing 4 mol of glycerol (Polyglyceryl-4 Oleyl Ether), oleyl alcohol containing 2 mol of glycerol (Polyglyceryl-2 Oleyl Ether), cetearyl alcohol containing 2 mol of glycerol, cetearyl alcohol containing 6 mol of glycerol, oleocetyl alcohol containing 6 mol of glycerol, or octadecanol containing 6 mol of glycerol; polyoxyethylenated fatty esters such as the adducts of ethylene oxide with esters of lauric acid, palmitic acid, stearic acid or behenic acid, and mixtures thereof, for example those containing from 9 to 100 oxyethylene units such as PEG-9 to PEG-50 laurate, PEG-9 to PEG-50 palmitate, PEG-9 to PEG-50 stearate, PEG-9 to PEG-50 palmitostearate, PEG-9 to PEG-50 behenate, polyethylene glycol 100 EO monostearate (PEG-100 stearate); glyceryl stearate (glyceryl mono-, di- and/or tristearate); glyceryl ricinoleate; sorbitan palmitate; sorbitan isostearate; sorbitan stearate; sorbitan palmitate; sorbitan trioleate; alkylglucose sesquistearates such as methylglucose sesquistearatel alkylglucose palmitates such as methylglucose or ethylglucose palmitate, etc., or combinations of two or more thereof may be chosen.

If present, the total amount of nonionic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of nonionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one nonionic surfactant, and have a total amount of nonionic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In at least some embodiments, the compositions are free or substantially free of nonionic surfactants.

### Cationic Surfactants

In at least some embodiments, the compositions comprise at least one cationic surfactant, although in at least some embodiments the compositions are free or essentially free of cationic surfactants. Exemplary and non-limiting cationic surfactants include cationic amine-based or quaternary ammonium-based compounds.

For example, cationic surfactants may be chosen from alkylpyridinium salts, ammonium salts of imidazoline, diquaternary ammonium salts, and ammonium salts containing at least one ester function.

The surfactants may be, for example, the salts (chloride or methyl sulfate) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

Other suitable cationic surfactants are esterquats which are quaternary ammonium compounds having fatty acid chains containing ester linkages, such as, for example, dibehenoylethyl dimonium chloride, dipalmitoylethyl dimonium chloride, distearoylethyl dimonium chloride, ditallowoyl PG-dimonium chloride, dipalmitoylethyl hydroxyethylmonium methosulfate, distearoylethyl hydroxyethylmonium methosulfate, or mixtures thereof.

Additional non-limiting examples of useful cationic surfactants include brassicamidopropyl dimethylamine, behentrimonium chloride, cetrimonium chloride, behenalkonium chloride, benzethonium chloride, cetylpyridinium chloride, behentrimonium chloride, lauralkonium chloride, cetalkonium chloride, cetrimonium bromide, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, stearamidopropyl dimethylamine, or combinations thereof.

If present, the total amount of cationic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of cationic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one cationic surfactant, and have a total amount of cationic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In some embodiments, however, the compositions are free or essentially free of cationic surfactants.

### Fatty Compounds

Optionally, compositions according to the disclosure may include at least one fatty compound, although in at least some embodiments the compositions are free or essentially free of fatty compounds. In certain embodiments, the at least one fatty compound may be chosen from lower alkanes, fatty alcohols, fatty acids, esters of fatty acids, esters of fatty alcohols, oils such as mineral, vegetable, animal, silicone and non-silicone oils, silicone and non-silicone waxes, or combinations of any two or more thereof. In some embodiments, the compositions comprise at least one fatty compound of natural origin. In some embodiments, the compositions are free or essentially free of fatty compounds that are not of natural origin. In some embodiments, the compositions are free or essentially free of silicone fatty compounds.

If present, silicone oils may, for example, be chosen from polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyl-diphenyl)trisiloxanes, or (2-phenylethyl)trimethyl-siloxysilicates. Non-limiting examples of silicone oils include dimethicone, amodimethicone, cyclomethicone, polysilicone-11, phenyl trimethicone, trimethylsilylamodimethicone, and stearoxytrimethylsilane. For example, the composition may comprise at least one silicone chosen from amodimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, Dimethicone PEG-8 Benzoate, Dimethicone PEG-7 Phosphate, Dimethicone PEG-8 Phosphate, Dimethicone PEG-10 Phosphate, or a combination of two or more thereof.

In some embodiments, the compositions comprise a silicone oil component that comprises, consists essentially of, or consists of dimethicone, amodimethicone, or a combination thereof. In some embodiments, the compositions are free or substantially free of silicone oils and/or silicone waxes.

In some embodiments, compositions according to the disclosure may include at least one fatty compound chosen from fatty alcohols. Herein, “fatty alcohol” can refer to any alcohol with a carbon chain of C5 or greater, such as, for example, C8 or greater, C10 or greater, or C12 or greater, such as from 6 to 30 carbon atoms or from 8 to 30 carbon atoms. The fatty alcohols may be alkoxylated or non-alkoxylated, saturated or unsaturated, and linear or branched. Non-limiting examples of fatty alcohols include arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or combinations of two or more thereof. In some preferred embodiments, the compositions comprise a fatty alcohol component that comprises, consists essentially of, or consists of cetyl alcohol, stearyl alcohol, cetearyl alcohol, or combinations thereof.

Useful and non-limiting fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, or combinations thereof.

In some embodiments, fatty acid esters or fatty alcohol esters may be chosen. For example, esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10 may be used. In other embodiments, esters of C4-C22 dicarboxylic or tricarboxylic acids and of C1-C22 alcohols and esters of mono-, di- or tricarboxylic acids and of C2-C26 di-, tri-, tetra-, or pentahydroxy alcohols may also be used. As non-limiting examples, isostearyl lactate, lauryl lactate, linoleyl lactate, oleyl lactate, (iso) stearyl octanoate, isocetyl octanoate, octyl octanoate, cetyl octanoate, decyl oleate, isocetyl isostearate, isocetyl laurate, isocetyl stearate, isodecyl octanoate, isodecyl oleate, isononyl isononanoate, isostearyl palmitate, methyl acetyl ricinoleate, myristyl stearate, octyl isononanoate, 2-ethylhexyl isononanoate, octyl palmitate, octyl pelargonate, octyl stearate, octyldodecyl erucate, oleyl erucate, ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate, dioctyl malate, hexyl laurate, 2-hexyldecyl laurate, diethyl sebacate, diisopropyl sebacate, diisopropyl adipate, di-n-propyl adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, and/or polyethylene glycol distearates may be chosen. In a preferred embodiment, the composition comprises at least one fatty acid ester and/or fatty alcohol ester, for example pentaerythrityl tetraisostearate.

Non-limiting examples of waxes that can be used include beeswax, hydrogenated alkyl olive esters, carnauba wax, candelilla wax, ouricoury wax, Japan wax, cork fibre wax or sugar cane wax, rice wax, rice bran wax, montan wax, paraffin wax, lignite wax or microcrystalline wax, ceresin or ozokerite, palm kernel glycerides/hydrogenated palm glycerides, palm butter, sumac wax, citrus Aurantium dulcis (orange) peel wax, Theobroma grandiflorum seed butter, Helianthus annuus (sunflower) seed wax, siliconyl candellila wax, Chinese wax, cetyl palmitate, lanolin, shellac, spermaceti, cetyl esters, hydrogenated castor wax; triglyceride esters such as tribehenin (glyceryl tribehenate); synthetic waxes such as those of the hydrocarbon type and polyethylene waxes obtained from the polymerization or copolymerization of ethylene, polypropylene waxes, or combinations of two or more of any of these waxes. However, in some embodiments the compositions are free or essentially free of waxes.

In some embodiments, the composition may include one or more fatty compounds chosen from oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C5-C9 esters, trimethylolpropane polyesters, propylene glycol polyesters, or polyesters of hydrogenated castor oil), perfluorinated and/or organofluorinated oils, fluorosilicone oils, or combinations of two or more thereof. In some embodiments, the compositions comprise at least one oil, e.g. mineral oil.

If present, the total amount of fatty compounds in the composition may range from about 0.01% up to about 15%, such as up to about 12%, up to about 10%, up to about 9%, up to about 8%, up to about 7%, up to about 6%, up to about 5%, or up to about 4% by weight, relative to the total weight of the composition. For example, the total amount of fatty compounds may range from about 0.1% to about 12%, from about 0.25% to about 10%, from about 0.5% to about 8%, from about 0.75% to about 6%, or from about 1% to about 5% by weight, relative to the total weight of the composition. In at least some embodiments, however, the compositions are free or essentially free of fatty compounds.

### Thickening Agents

Compositions according to the disclosure optionally comprise at least one thickening agent. Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, celluloses such as hydroxyethylcellulose and hydroxypropylcellulose, and guars such as hydroxypropyl guar.

In some embodiments, the thickening agent may be chosen from associative thickening polymers such as anionic associative polymers, amphoteric associative polymers, cationic associative polymers, or nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may range from about 0.001% to about 5%, such as from about 0.01% to about 4%, from about 0.1% to about 3.5%, from about 0.2% to about 3%, from about 0.3% to about 2.5%, from about 0.4% to about 2%, from about 0.5% to about 1.5%, or from about 0.5% to about 1% by weight, relative to the total weight of the composition.

### Amino Acids and Amino Sulfonic Acids

The compositions may optionally comprise at least one amino acid and/or amino sulfonic acid, although in at least some embodiments the compositions are free or essentially free of amino acids and/or amino sulfonic acids. Salts of amino acids and amino sulfonic acids may also be chosen, and are expressly included whether or not so stated.

Amino acids that may be chosen include, for example, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Exemplary amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, and amino hexyl benzene sulfonic acid. In some embodiments, the hair treatment compositions comprise at least one amino acid and at least one amino sulfonic acid. In some embodiments, the composition comprises arginine, serine, betaine, and/or taurine, for example arginine, betaine, or a combination thereof. For example, in some embodiments, arginine, serine, betaine, and/or taurine are the only amino acids and amino sulfonic acids present in the compositions, and in some embodiments, arginine and/or betaine are the only amino acids and amino sulfonic acids present in the compositions.

If present, the total amount of amino acids and/or amino sulfonic acids may range from about 0.01% to about 10%, such as from about 0.1% to about 8%, or from about 0.5% to about 6% by weight, relative to the total weight of the composition. For example, in some embodiments, the total amount of amino acid(s) and/or amino sulfonic acid(s) may range from about 0.5% to about 5%, from about 0.5% to about 4%, from about 0.5% to about 3%, from about 0.5% to about 2.5%, from about 0.5% to about 2%, from about 0.5% to about 1.5%, from about 0.5% to about 1%, from about 0.75% to about 5%, from about 0.75% to about 4%, from about 0.75% to about 3%, from about 0.75% to about 2.5%, from about 0.75% to about 2%, from about 0.75% to about 1.5%, from about 0.75% to about 1%, from about 1% to about 5%, from about 1% to about 4%, from about 1% to about 3%, from about 1% to about 2.5%, from about 1% to about 2%, or from about 1% to about 1.5% by weight, relative to the total weight of the composition. In some embodiments, the total amount of amino acid(s) and/or amino sulfonic acid(s) may be about 0.25%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including all ranges and subranges using any of the foregoing as upper and lower limits.

### Auxiliary Components

Compositions according to the disclosure may optionally include one or more auxiliary components. Non-limiting examples include preservatives, fragrances such as parfum, pH adjusters (e.g. citric acid, maleic acid), salts, antioxidants, vitamins (e.g. ascorbic acid, tocopherol), vitamin derivatives, botanical extracts, buffers, sequestering agents, and the like. In some embodiments, the compositions are free or essentially free of any of the aforementioned, e.g. the compositions may be free or essentially free of antioxidants, free or essentially free of ascorbic acid, etc.

The total amount of auxiliary components, if present, typically ranges from about 0.01% to about 15% by weight, based on the total weight of the composition. For example, in some embodiments the individual amounts of each component or the total amount of components may range from about 0.1% to about 10%, about 0.1% to about 8%, about 0.1% to about 5%, about 0.1% to about 4%, about 0.1% to about 3%, about 0.1% to about 2%, about 0.25% to about 10%, about 0.25% to about 8%, about 0.25% to about 5%, about 0.25% to about 4%, about 0.25% to about 3%, about 0.25% to about 2%, about 0.5% to about 10%, about 0.5% to about 8%, about 0.5% to about 5%, about 0.5% to about 4%, about 0.5% to about 3%, about 0.5% to about 2%, about 0.75% to about 10%, about 0.75% to about 8%, about 0.75% to about 5%, about 0.75% to about 4%, about 0.75% to about 3%, or about 0.75% to about 2% by weight, based on the total weight of the composition.

In some embodiments, the compositions may be free or essentially free of one or more of the following: ascorbic acid and salts thereof, dyes or pigments for coloring the hair, persulfates, peroxides, and/or ammonia. In yet other embodiments, the compositions may comprise less than 5%, less than 4.5%, less than 4%, less than 3.5%, less than 3%, less than 2.5%, less than 2%, less than 1.5%, less than 1.25%, less than 1%, less than 0.75%, less than 0.5%, less than 0.4%, less than 0.3%, less than 0.2%, less than 0.1%, less than 0.05%, or less than 0.01% of a total amount of ascorbic acid and salts thereof, persulfates, peroxides, or ammonia, or of a total amount of any combination of two or more thereof.

In some embodiments, the compositions may be free or essentially free of components that are traditionally used to remove hair color, such as oxidizing agents and/or reducing agents. By way of example, the composition may be free or essentially free of cysteine, homocysteine, thiolactic acid, thiosalicylic acid, thioglycolic acid, glutaric acid, glyoxylic acid, glycolic acid, citric acid, ascorbic acid, salts thereof, and/or derivatives thereof. In some embodiments the compositions are free or essentially free of thiol-based color-removing agents such as thiolactic acid, thiosalicylic acid, thioglycolic acid, and/or salts thereof. In various embodiments, if any of the aforementioned components are included, the compositions may comprise less than 5%, less than 4.5%, less than 4%, less than 3.5%, less than 3%, less than 2.5%, less than 2%, less than 1.5%, less than 1.25%, less than 1%, less than 0.75%, less than 0.5%, less than 0.4%, less than 0.3%, less than 0.2%, less than 0.1%, less than 0.05%, or less than 0.01% of any of these components or of a total amount of these components combined.

The compositions may be in any suitable form. For example, the compositions may be a liquid, a gel, a gel cream, a cream, a serum, etc. Advantageously, the compositions can be used as a one-part hair color removing composition, as compared to known systems for removing color from hair that require the application of multiple compositions to effectively remove color from the hair. In particular, the compositions are stable for at least 24 hours, at least 48 hours, at least 72 hours, at least one week, at least two weeks, at least four weeks, at least two months, at least six months, or at least one year, unlike known hair remover compositions that require mixture with water or a developer immediately before use due to their unstable nature.

In various embodiments, the viscosity of the compositions may be greater than about 500 cPs and less than about 2500 cPs, for example may range from about 800 to about 2000 cPs, such as from about 1000 to about 1800 cPs, or from about 1200 to about 1600 cPs when measured using a Rheomat with an M3 spindle at about 25° C., for a time period of about 30 seconds.

The pH of the compositions may range from about 5 to about 11, such as from about 6 to about 10, from about 6.5 to about 9.5, from about 7 to about 9, from about 7.5 to about 8.5, or may be about 8. For example, the pH of the composition may be about 6, about 6.5, about 7, about 7.5, about 8, about 8.5, about 9, about 9.5, or about 10.

### II. Methods

In various embodiments, the disclosure relates to methods for altering the color, tone, and/or shade of hair. The compositions and methods can be used to remove some or all of certain semi-permanent dyes, while having minimal or no impact on other dyes. As such, the compositions can be targeted to remove certain dyes from the hair to achieve the consumer's desired result. For example, the methods may comprise methods of increasing the warmth of a hair color, for example by targeting dyes for removal that cause a color to be cooler. As a further example, the methods may comprise methods of increasing cool tones by targeting dyes for removal that cause a color to be warmer.

It is understood by those skilled in the art that the color, tone, and shade of hair are evaluated using the CIE L*a*b* system. In this system, the change in color, tone, and/or shade is determined by evaluating the color of hair after treatment (L*₂, a*₂, b*₂) compared to the color of the hair before treatment (L*₁, a*₁, b*₁). The overall change in color (ΔE) is defined as:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

In this system, the three parameters represent, respectively, the color intensity (L*), the green/red color axis (a*) (for example, higher a* is more red) and the blue/yellow color axis (b*) (for example, higher b* is more yellow). The higher the value of L*, the lighter the color, the higher the value of a*, the redder the color, and the higher the value of b*, the yellower the color. Additionally, the higher the value for ΔE, the greater the difference in color of treated hair relative to the color of the hair prior to treatment. Thus, methods may comprise altering the color, tone, and/or shade of hair by varying L*2, a*2, and/or b*2 values, and/or by increasing the ΔE value relative to the hair prior to treatment.

The compositions and methods are thus able to achieve a desired hair color, tone, and/or shade even without, or with minimal, application of, subsequent hair coloring compositions. However, although the hair color, tone, and/or shade can be modified using compositions and methods according to the disclosure, in at least some embodiments the methods comprise an additional step of coloring or toning the hair after the hair is treated according to the disclosure.

The disclosure also relates to methods of removing artificial color from keratin fibers, especially hair that has been previously dyed with a semi-permanent dye. In particular, the compositions and methods according to the disclosure are particularly useful for removing darker colors from hair that may be otherwise difficult to remove with traditional hair-color removing compositions that do not use oxidizing agents. By way of non-limiting example, darker colors that can be removed in methods according to the disclosure include blacks, ash browns, browns, ash mochas, natural ash (blue or green), ash violets, violets, red violets, reds, red mochas, red browns, mahogany, red coppers, coppers, and golds. The methods also comprise removing semi-permanent color from hair that has been dyed with both semi-permanent and permanent (oxidative) dyes. The methods permit removal of the semi-permanent dyes without removing the color imparted by the oxidative dyes.

The methods comprise applying a composition according to the disclosure onto the keratin fibers, e.g. hair that has been previously-dyed with at least one semi-permanent hair dye compound, optionally leaving the composition on the keratin fibers for a period of time (“processing time” or “leave-in period”), and subsequently rinsing or otherwise removing the composition from the keratin fibers. Thus, the methods may comprise a two-step method, which is simpler and more effective than known three- or more step methods where multiple compositions are applied to the hair for color removal before the hair is rinsed.

The appropriate leave-in period will be determined based on the color to be removed, and may in various embodiments last up to about 60 minutes, up to about 90 minutes, up to about 120 minutes, up to about 150 minutes, up to about 180 minutes, etc., such as from about 10 minutes to about 6 hours, from about 30 minutes to about 3 hours, from about 45 minutes to about 150 minutes, or from about 60 minutes to about 2 hours. It may be advantageous, in various embodiments, to leave the color-removing composition on the hair for at least about 30 minutes, and up to about 3 hours. For example, the leave-in period may range from about 30 minutes to about 180 minutes, from about 45 minutes to about 150 minutes, or from about 60 minutes to about 120 minutes. Optionally, the hair may be covered, such as with a processing cap or aluminum foil, for at least a portion of the leave-in period.

In some embodiments, the hair to which a composition according to the disclosure has been applied may be heated during the leave-in period. For example, the hair may be heated using a hair dryer, hood dryer, or any other method for at least portion of the leave-in period. The hair may be heated using temperatures above room temperature, for example ranging from about 27° C. to about 90° C., from about 30° C. to about 80° C., from about 35° C. to about 70° C., or from about 40° C. to about 50° C. For example, an exemplary heating temperature range may be about 40° C. to about 45° C., such as about 40° C. to about 43° C.

In various embodiments, it may be advantageous to heat the treated hair for at least a portion of the leave-in period, with no heating for at least a portion of the leave-in period. By way of example, after the color-removing composition is applied to the hair, the treated hair may be left to process for a period of time at room temperature, followed by a period of time with application of heat. As an alternate example, after the color-removing composition is applied to the hair, the treated hair may be heated, followed by a period of time when the hair processes at room temperature. Further variations on such methods of alternating heating the treated hair and allowing the treated hair to process at room temperature may be used, such as, for example, heating followed by room temperature followed by heating, or processing at room temperature followed by heating followed by processing at room temperature, and so on.

Surprisingly, the compositions and methods according to the disclosure effectively remove semi-permanent color from hair without the use of traditional color-removing agents such as oxidizing agents and reducing agents, without the damage that is typically associated with color removing compositions containing oxidizing agents, and without the odor that is typically associated with color removing compositions containing thiol-based compounds.

In some embodiments, it may be advantageous to perform the methods described herein more than one time, in order to obtain the desired level of color removal. Because the compositions minimize damage to the hair and have decreased odor compared to known color-removal compositions, consumers are able to achieve the desired color removal without the disadvantages associated with such known compositions.

It should be understood that compositions according to the disclosure are not compositions for coloring the hair. As such, the compositions are free or essentially free of dyes or pigments for coloring the hair, including semi-permanent and oxidative dye compounds. The compositions may optionally comprise a dye or pigment for coloring the composition, but it will be understood that the amount and/or type of dye or pigment will be chosen so that it does not impart color to the hair. Likewise, the methods according to the disclosure are not methods for imparting color to the hair.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means+/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the phrase “applying a composition onto keratin fibers” and variations thereof are intended to mean contacting the keratin fibers such as hair with at least one of the compositions of the disclosure, in any manner. It may also mean contacting the keratin fibers with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

Unless otherwise defined for any specific embodiment, the term “substantially free” or “essentially free” as used herein means that there is less than about 5% by weight of a specific material added to a composition, based on the total weight of the composition. For example, the compositions may include less than about 4%, less than about 3%, less than about 2%, less than about 1.5%, less than about 1%, less than about 0.5%, less than about 0.1%, less than about 0.01%, less than about 0.001%, or less than about 0.0001% of the specified material. As such, it is contemplated that any component described herein for use in the compositions can be present in the compositions in amounts less than about 5%, less than about 4%, less than about 3%, less than about 2%, less than about 1%, less than about 0.5%, less than about 0.1%, less than about 0.01%, less than about 0.001%, or less than about 0.0001%, and the composition will be considered “substantially free” of such material. A composition that is “free” of a component is understood to contain none of the specified component. However, it is understood that the terms “free” and “substantially free” refer to the amount of a component added to the composition, without including an amount of the component present in the composition as a minor component in a raw material. For example, a composition that is “free” of waxes may not have wax included as an intended component but may nevertheless contain a pigment that is coated with a wax, as such wax would be considered a minor component of the pigment material and would not be expected to provide benefits to the composition that would be expected by including a wax per se as an intended component.

As used herein, the terms “treat,” “treated,” “treatment,” and variations thereof is not intended to be limiting, but rather is merely intended to indicate that one or more compositions is applied to the hair, and optionally removed from the hair. For example, hair that is “treated” with a composition according to the disclosure may have had the composition applied, and/or may have had the composition applied and removed, e.g. by rinsing or towel drying. As a further example, hair that is “treated” with a composition according to the disclosure may have had the composition applied, and/or may have had the composition applied and rinsed from the hair.

As used herein, “altering the color of hair,” “modifying the color of hair,” and variations thereof means that the color, tone, and/or shade of hair is changed by removing previously-applied hair dyes from the hair. Further, the terms “color removal,” “hair color-removing,” and variations thereof should be understood to refer to removal of some or all of previously-applied hair dyes from the hair. It should be understood that removal of artificial color can include removal of all color, or can include removal of various tones of color. Therefore, in some embodiments, overall change in the hair color will be observed (e.g. greater ΔE), while in other embodiments even if the overall color change is not considered significant, variation in one or more of the L*, a*, or b* values will be observed.

As used herein, “artificial color” means color that has been imparted to the hair by means of prior application of a composition capable of changing the color of hair, for example with permanent, semi-permanent, or demi-permanent hair dye compositions. In various embodiments the artificial color that is removed from the hair is color that has been imparted to the hair by one or more semi-permanent dye compounds.

As used herein, the terms “odor-free,” “minimal odor,” “no odor,” “reduced odor,” or the like are intended to mean the odor that is typically associated with color removing compositions containing thiolactic acid color removers, which odor hair color professionals and consumers typically find pungent and/or offensive.

The term “stable” as used herein means that no phase separation is observed when the composition is stored at room temperature and pressure for the specified period of time.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention.

### EXAMPLES

The following Examples are intended to be non-limiting and explanatory in nature only. In the Examples, amounts are expressed in percentage by weight (wt %) of active materials, relative to the total weight of the composition, unless otherwise indicated.

In these Examples, the change in the color of hair is evaluated with the CIE L*a*b* system using Colorshot MS, where the change is determined by evaluating the color of the hair after treatment (L*₂, a*₂, b*₂) compared to the color of the hair before treatment (L*₁, a*₁, b*₁). The change in color (ΔE) is calculated as follows:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} .$$

### Example 1—Color Altering Compositions

Compositions 1A-1E according to the disclosure and comparative compositions C1-C3 were prepared as shown in Tables 1A and 1B. Table 1C shows comparative composition C4, which is a commercially available hair color removing composition.

TABLE 1A
Hair Color Altering Compositions

| | 1A | 1B | 1C | 1D | 1E |
|---|---|---|---|---|---|
| HECTORITE | | | | 10.0 | |
| MAGNESIUM ALU- | 10.0 | | | | |
| MINUM SILICATE | | | | | |
| BENTONITE | | 10.0 | | | 10.0 |
| KAOLIN | | | 20.0 | | |
| ARGININE | | | | | 3.0 |
| WATER | QS | QS | QS | QS | QS |
| | to 100 | to 100 | to 100 | to 100 | to 100 |

TABLE 1B
Comparative Hair Color Altering Compositions

| | C1 | C2 | C3 |
|---|---|---|---|
| MAGNESIUM ALUMINUM SILICATE | | 10.0 | |
| KAOLIN | | | 0.05 |
| KETOGLUTARIC ACID | 10.0 | 10.0 | |
| THIOLACTIC ACID | 5.0 | 5.0 | |
| AMMONIUM CHLORIDE | | | 5.0 |
| STRONTIUM PEROXIDE | | | 11.5 |
| SODIUM BENZOATE | | | 0.3 |
| DIETHYLHEXYL SODIUM | | | 1.7 |
| SULFOSUCCINATE | | | |
| EDTA | | | 1.0 |
| SODIUM METASILICATE | | | 9.0 |
| SODIUM LAURYL SULFATE | | | 14.0 |
| POTASSIUM PERSULFATE | | | 20.0 |
| SODIUM PERSULFATE | | | 33.0 |
| CELLULOSE GUM | | | 4.0 |
| WATER | QS to 100 | QS to 100 | |

TABLE 1C
Commercial Hair Color Remover Composition
C4 - Ingredients listed on package

Ascorbic Acid, Bentonite, Xanthan Gum, Disodium EDTA,
Polyquaternium-10, Sodium Cocoyl Isethionate, Sodium
Isethionate, Sodium Gluconate

### Example 2—Evaluation of Removal of Semi-Permanent Hair Color

The following studies were performed to evaluate the efficacy of compositions according to the disclosure to remove color and to alter the color, tone, and/or shade of hair that has previously been dyed with semi-permanent hair dyes. The dyes used in the studies are shown in Table 2A.

TABLE 2A
Dyes

| Dye | D1 | D2 | D3 | D4 | D5 |
|---|---|---|---|---|---|
| Basic red 51 (azo) | X | X | | X | |
| Basic orange 31 | | | | X | X |
| Basic yellow 29 | | | X | | |
| (azomethine) | | | | | |
| Basic yellow 87 | | | | X | |
| Basic blue 99 | | | | X | |
| HC blue 15 | | X | | X | |
| (triarylmethane) | | | | | |
| Basic blue 3 | | | X | | X |
| (oxazine) | | | | | |
| Basic violet 2 | | X | | | X |
| (triarylmethane) | | | | | |
| Solvent violet 13 | | | | | X |
| Basic brown 17 | | | | X | |

### Example 2A—Removal of Semi-Permanent Hair Dyes: 60 Minutes, Single Application

Three sets of hair swatches were colored with one of three different commercially-available semi-permanent hair dye compositions (D1-D3) (21 swatches total, 9 swatches for D1 and 6 swatches for each of D2 and D3). Prior to the dyeing step, all but one of the swatches were platinum-bleached, with the last swatch being permed, ˜90% grey hair. The dye compositions were applied to dry swatches at a rate of about 3 grams per gram of hair. The swatches were left to process at 27° C. for 35 minutes, rinsed, and dried. Once the swatches were dry, the swatches were treated as follows.

Four of the swatches in each set were treated with compositions according to the disclosure. One of compositions 1A-1C or 1E was applied to one swatch from each set at a rate of about 2 grams per gram of hair. After the swatches were allowed to process for 60 minutes at 33° C., the swatches were rinsed, followed by three shampoo/rinse cycles using a commercial shampoo, and then dried.

One swatch per set was treated with comparative composition C3. Just prior to use, 28 grams of composition C3 were mixed with 75 mL of a commercially-available 30V developer composition. The mixture was applied to the swatches at a rate of about 2 grams per gram of hair. After the swatches were allowed to process for 60 minutes at 33° C., the swatches were rinsed, followed by three shampoo/rinse cycles using a commercial shampoo, and then blow-dried.

Three swatches from the set dyed with composition D1 were treated with comparative compositions C1, C2, or C4, as follows. Just prior to use, 20 grams of composition C4 were mixed with 4 ounces of room temperature water and the mixture was shaken as instructed on package directions, to form a mixture having a pH of about 2.5. About 2 grams of either the C4 mixture, composition C1, or composition C2 were applied to one of the swatches per gram of hair, with the C4 mixture applied to the permed swatch. The swatches treated with compositions C1 and C2 were then allowed to process 60 minutes, and the swatch treated with the C4 mixture was allowed to process for 45 minutes, all at a temperature of about 33° C. Once the processing time ended, the swatches were rinsed, followed by three shampoo/rinse cycles using a commercial shampoo, and then blow-dried.

One swatch per set had no color-removing composition applied, and was the color control.

The color of each of the swatches was then evaluated with the CIE L*a*b* system, to evaluate the effectiveness of color removal after 60 minutes with one application of the hair color removing compositions. Table 2B shows the ΔE values for the treated swatches, relative to the color control, and Table 2B shows the L*, a*, and b* results.

TABLE 2B
Measured ΔE Results: 60 Minutes, Single Application

| DYE | Control | 1A | 1B | 1C | 1E | C3 | C1 | C2 | C4 |
|---|---|---|---|---|---|---|---|---|---|
| D1 | — | 42.16 | 45.63 | 32.34 | 53.44 | 37.81 | 10.74 | 38.84 | 14.16 |
| D2 | — | 42.04 | 42.20 | 27.92 | 41.42 | 65.26 | — | — | — |
| D3 | — | 32.90 | 43.16 | 24.09 | 48.47 | 57.36 | — | — | — |

TABLE 2C
Measured L*a*b* Results: 60 Minutes, Single Application

| | D1 | | | D2 | | | D3 | | |
|---|---|---|---|---|---|---|---|---|---|
| | L* | a* | b* | L* | a* | b* | L* | a* | b* |
| C1 | 28.48 | 54.67 | 33.62 | — | — | — | — | — | — |
| C2 | 52.19 | 28.60 | 19.28 | — | — | — | — | — | — |
| C4 | 28.20 | 50.90 | 32.70 | — | — | — | — | — | — |
| C3 | 57.34 | 41.43 | 24.37 | 66.07 | 4.45 | 22.69 | 64.71 | −10.54 | 24.51 |
| 1A | 53.91 | 24.68 | 20.1 | 51.07 | 8.79 | 3.62 | 50.24 | −29.00 | 9.56 |
| 1B | 56.50 | 21.73 | 21.64 | 48.82 | 9.80 | 6.48 | 54.21 | −17.57 | 16.35 |
| 1C | 46.77 | 41.52 | 11.77 | 40.11 | 13.41 | −6.03 | 43.50 | −37.81 | 2.52 |
| 1E | 60.86 | 14.01 | 24.63 | 47.70 | 8.29 | 4.85 | 57.41 | −13.80 | 19.06 |

The results in Table 2B demonstrate that compositions according to the disclosure are effective in removing semi-permanent hair dyes from the hair. For example, for dye composition D1, compositions 1A, 1B, and 1E resulted in greater overall color change (greater ΔE) compared to all of C₁-C₄, and 1C was better than C1 and C4.

The results in Table 2C show that compositions according to the disclosure can be used to alter the color, tone, and/or shade of hair in a controlled manner. For example, as the results for dye composition D1 show, although composition 1C had a lower overall change in color than composition C2, relative to composition C2 composition 1C provided a more reddish tone (higher a* value). Similarly, as the results for dye composition D2 show, although composition 1B provides a lighter (higher L* value) than composition 1C, composition 1C provides a more reddish tone (higher a* value) than composition 1B.

These data demonstrate that the compositions and processes according to the disclosure can be used to modify hair color in a targeted manner, unlike traditional hair color-removing compositions. Compositions according to the disclosure have the unique ability to fade-on-tone and deliver the desired color, tone, and/or shade, even in the absence of subsequent hair color applications.

### Example 2B—Removal of Semi-Permanent Hair Color: 120 Minutes, Single Application

The process of Example 2A was repeated with composition 1A but with a longer processing time (120 minutes) for swatches dyed with semi-permanent hair dye compositions D1 and D3. The ΔE values for the treated swatches, relative to the color control are shown in Table 2D, and the L*, a*, and b* values for the treated swatches are shown in Table 2E.

TABLE 2D
Measured ΔE Results: 120 Minutes, Single Application

| DYE | CONTROL | 1A | C3 |
|---|---|---|---|
| D1 | — | 49.42 | 37.81 |
| D3 | — | 54.00 | 57.36 |

TABLE 2E
Measured L*a*b* Results: 120 Minutes, Single Application

| | D1 | | | D3 | | |
|---|---|---|---|---|---|---|
| | L* | a* | b* | L* | a* | b* |
| 1A | 58.79 | 18.32 | 22.07 | 60.12 | −8.44 | 19.63 |

The results in Tables 2D and 2E confirm that compositions according to the disclosure can be used to effectively remove semi-permanent color from the hair in order to alter the color, tone, and/or shade of the hair.

### Example 2C—Removal of Semi-Permanent Hair Color: 60 Minutes, Single Application

The process of Example 2A was repeated with composition 1A but using swatches dyed with semi-permanent hair dye composition D4 or D5. The ΔE values for the treated swatches, relative to the color control are shown in Table 2F, and the L*, a*, and b* values for the treated swatches are shown in Table 2G.

TABLE 2F
Measured ΔE Results: 60 Minutes, Single Application

| DYE | CONTROL | 1A | C3 |
|---|---|---|---|
| D4 | — | 36.20 | 47.48 |
| D5 | — | 30.28 | 57.82 |

TABLE 2G
Measured L*a*b* Results: 60 Minutes, Single Application

| | D4 | | | D5 | | |
|---|---|---|---|---|---|---|
| | L* | a* | b* | L* | a* | b* |
| 1A | 56.58 | −6.32 | 16.41 | 23.96 | 6.12 | −27.89 |

The results in Tables 2F and 2G further confirm that compositions according to the disclosure can be used to effectively remove semi-permanent color from the hair in order to alter the color, tone, and/or shade of the hair.

### Example 2D—Removal of Semi-Permanent Hair Color: 60 Minutes, Repeated Application

The process of Example 2A was repeated with various combinations of color-removing compositions and semi-permanent dye compositions. However, in this Example after the end of the 60 minute processing time for the color-removing composition the hair was treated by reapplying the same color-removing composition and allowing the swatch to process for another 60 minutes. The ΔE values for the treated swatches, relative to the color control are shown in Tables 2H-1, 2H-2, 2H-3, and 2H-4.

TABLE 2H-1
Measured ΔE Results: 60 Minutes, Repeated Application

| DYE | CONTROL | 1A | C3 |
|---|---|---|---|
| D2 | — | 53.05 | 65.26 |

TABLE 2H-2
Measured ΔE Results: 60 Minutes, Repeated Application

| DYE | CONTROL | 1B | C3 |
|---|---|---|---|
| D3 | — | 55.32 | 57.36 |

TABLE 2H-3
Measured ΔE Results: 60 Minutes, Repeated Application

| DYE | CONTROL | 1B | C3 |
|---|---|---|---|
| D4 | — | 39.22 | 47.48 |

TABLE 2H-4
Measured ΔE Results: 60 Minutes, Repeated Application

| DYE | CONTROL | 1B | C3 |
|---|---|---|---|
| D5 | — | 33.89 | 57.82 |

The results in Tables 2H-1 through 2H-4 similarly confirm that compositions according to the disclosure can be used to effectively remove semi-permanent color from the hair in order to alter the color, tone, and/or shade of the hair.

The studies in Example 2 show that compositions having clay compounds as color-removing agents are effective at removing semi-permanent hair dyes.

### Example 3—Evaluation of Color Removal on Oxidative Dyes

The following studies were performed to evaluate color removal benefits using compositions according to the disclosure on oxidative hair dyes.

Three swatches of hair were colored with a commercially-available oxidative hair dye composition. The oxidative hair dye composition was mixed in a 1:1 ratio with 20V hydrogen peroxide developer to form a hair dye mixture, and the hair dye mixture was applied to the swatches at a rate of about 3 grams per gram of hair. The swatches were left to process at 27° C. for 35 minutes, rinsed, shampooed with a commercial shampoo, and rinsed again. The swatches were then subjected to four additional shampoo/rinse cycles and allowed to air dry.

Once the swatches were dry, two of the swatches were treated as follows. One swatch had composition 1A applied at a rate of about 2 grams per gram of hair left to process for 60 minutes at 33° C. A second swatch had composition C1 applied at a rate of about 2 grams per gram of hair left to process for 30 minutes at 33° C. Once the processing time was complete, the swatches were rinsed for about 30 seconds, shampooed with a commercial shampoo, rinsed, shampooed again, rinsed again, and then blow-dried. One swatch per set had no color-removing composition applied, and was the color control.

The color of each of the swatches was then evaluated with the CIE L*a*b* system, to evaluate the effectiveness of color removal. Table 3 shows the ΔE values for the treated swatches, relative to the color control.

TABLE 3
Measured ΔE Results

| | ΔE |
|---|---|
| Control | — |
| C1 | 52.1 |
| 1A | 1.45 |

The results in Table 3A confirm that oxidative hair dye can be removed with composition C1 having thiolactic acid as a color remover, whereas composition 1A according to the disclosure provides almost no removal of the oxidative dyes.

Example 3 therefore demonstrates that compositions according to the disclosure can be used for removing semi-permanent hair dyes with minimal impact on color imparted by oxidative dyes.

### Example 4—Additional Compositions

Compositions 2A-2D can also be prepared as shown in Table 4, and are likewise expected to provide similar hair color removing and reduced damage benefits.

TABLE 4

| | 2A | 2B | 2C | 2D |
|---|---|---|---|---|
| HECTORITE | 4.0 | | | 5.0 |
| MAGNESIUM ALUMINUM | 6.0 | | 2.0 | 5.0 |
| SILICATE | | | | |
| BENTONITE | | 5.0 | | 5.0 |
| KAOLIN | | | 5.0 | 5.0 |
| ARGININE | | 2.5 | 1.0 | |
| BETAINE | 1.9 | | | |
| PROLINE | | | | 0.5 |
| XANTHAN GUM | 0.7 | 0.7 | | |
| HYDROXYPROPYL | | | 1.5 | 1.0 |
| CELLULOSE | | | | |
| SODIUM LAURYL SULFATE | 5.0 | 5.0 | | |
| SODIUM LAUROYL METHYL | | | 2.5 | 7.5 |
| ISETHIONATE | | | | |
| CITRIC ACID | | | 5.0 | |
| FATTY COMPOUNDS | | | 1.5 | 5.0 |
| (silicone oils or plant oils) | | | | |
| ADDITIVES (fragrances, pH | ≤5 | ≤5 | ≤5 | ≤5 |
| adjuster, vitamins, plant | | | | |
| extracts, preservatives, etc.) | | | | |
| SOLVENTS (water and non- | QS | QS | QS | QS |
| aqueous solvents) | to 100 | to 100 | to 100 | to 100 |

The above Examples demonstrate that compositions and methods according to the disclosure effectively remove semi-permanent hair dyes from hair, allowing the hair color, tone, and/or shade to be altered in a controlled manner.

These compositions and processes are unique in that they can provide a targeted method for removing hair color to provide consumers with choices in resulting color, even without the need to backfill the color to arrive at the desired result. The compositions and processes can be particularly useful when similar tonality, rather than an entirely different hair color, is desired, as the data in the above Examples demonstrate.

Moreover, the Examples show that the compositions are processes are particularly useful for removing darker colors, which tend to be more difficult to remove with color-removing agents that are not oxidizing agents.

1. A method for method for altering the color of hair, the method comprising applying to the hair a composition comprising:
a) at least one color-removing agent chosen from clay compounds;
b) at least one solvent; and
c) optionally at least one additional component chosen from surfactants, fatty compounds, thickeners, amino acids and/or salts thereof, amino-sulfonic acids and/or salts thereof, or combinations of two or more thereof,
wherein the total amount of clay compounds present in the composition ranges from about 1% to about 30% by weight, relative to the total weight of the composition.

2. The method according to claim 1, wherein the composition comprises (a) from about 5% to about 20% of clay compounds by weight, relative to the total weight of the composition.

3. The method according to claim 1, wherein the composition comprises no more than 0.5% of color-removing agents other than clay compounds.

4. The method according to claim 1, wherein the composition has a pH ranging from about 6 to about 10.

5. The method according to claim 1, wherein the composition further comprises at least one additional component chosen from surfactants, fatty compounds, thickeners, amino acids and/or salts thereof, amino-sulfonic acids and/or salts thereof, or combinations of two or more thereof.

6. The method according to claim 1, wherein the composition is left on the hair for a period of time ranging from about 30 minutes to about 180 minutes and then rinsed from the hair.

7. The method according to claim 6, wherein the hair to which the composition is applied is heated before the hair is rinsed.

8. The method according to claim 1, wherein the composition comprises:
a) at least one clay compound chosen from kaolin, hectorite, magnesium aluminum silicate, bentonite, or combinations of two or more thereof;
b) water; and
c) optionally at least one additional component chosen from surfactants, fatty compounds, thickeners, amino acids and/or salts thereof, amino-sulfonic acids and/or salts thereof, or combinations of two or more thereof,
wherein the total amount of clay compounds ranges from about 5% to about 15% by weight, relative to the total weight of the composition.

9. The method according to claim 8, wherein the composition comprises no more than 0.5% of color-removing agents other than clay compounds.

10. The method according to claim 8, wherein the composition is left on the hair for a period of time ranging from about 30 minutes to about 180 minutes and then rinsed from the hair.

11. A hair-color altering composition comprising:
a) at least one color-removing agent chosen from clay compounds;
b) at least one solvent; and
c) at least one additional component chosen from surfactants, fatty compounds, thickeners, amino acids and/or salts thereof, amino-sulfonic acids and/or salts thereof, or combinations of two or more thereof,
wherein the total amount of clay compounds ranges from about 5% to about 25% by weight, relative to the total weight of the composition.

12. The hair-color altering composition according to claim 11, comprising (a) at least one clay compound chosen from kaolin, hectorite, magnesium aluminum silicate, bentonite, or combinations of two or more thereof.

13. The hair-color altering composition according to claim 11, wherein the composition comprises no more than 0.5% of color-removing agents other than clay compounds.

14. The hair-color altering composition according to claim 11, wherein the composition has a pH ranging from about 6 to about 10.

15. The hair-color altering composition according to claim 11, comprising (b) water and at least one non-aqueous solvent.

16. The hair-color altering composition according to claim 11, wherein the composition is free of oxidizing agents and thiol-based compounds.

17. The hair-color altering composition according to claim 11, comprising at least one amino acid and/or salt thereof.

18. The hair-color altering composition according to claim 11, comprising at least one surfactant.

19. A hair-color altering composition comprising:
a) at least one hair-color removing agent;
b) water; and
c) at least one additional component chosen from surfactants, fatty compounds, thickeners, amino acids and/or salts thereof, amino-sulfonic acids and/or salts thereof, or combinations of two or more thereof,
wherein the hair-color removing agent comprises at least one clay compound chosen from kaolin, hectorite, magnesium aluminum silicate, bentonite, or combinations of two or more thereof,
wherein the total amount of clay compounds ranges from about 1% to about 30% by weight, relative to the total weight of the composition, and
wherein the composition has a pH ranging from about 6 to about 10.

20. The hair-color altering composition according to claim 19, wherein the composition has a pH ranging from about 7 to about 9.
